(19) United States
(12) Patent Application Publication
(10) Pub. No.: US 2004/0106564 A1
(43) Pub. Date: Jun. 3, 2004

(54) Use of slpi for treating chronic inflammatory intestinal diseases

(76) Inventor: Manfred Nilius, Magdeburg (DE)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/250,901

(22) PCT Filed: Dec. 11, 2001
(86) PCT No.: PCT/EP01/14518

(30) Foreign Application Priority Data
Jan. 17, 2001 (DE) 101 01 793.6

(51) Int. Cl. A61K 48/00

(52) U.S. Cl. 514/44

(57) ABSTRACT

The present invention relates to the use of secretory leucocyte protease inhibitor (SLPI), or a non-pathogenic microorganism capable of forming SLPI and containing a nucleic acid coding for SLPI, for the treatment of chronic inflammatory intestinal diseases of humans and animals, pharmaceutical compositions for oral or rectal administration which contain the effective material SLPI or SLPI-expressing microorganisms, and methods for the production of these pharmaceutical compositions.

### FIELD OF THE INVENTION

The present invention relates to the use of secretory leucocyte protease inhibitor (SLPI) or a non-pathogenic microorganism containing a nucleic acid coding for SLPI and capable of forming SLPI, for the treatment of chronic inflammatory intestinal diseases of humans and animals, pharmaceutical compositions for oral or rectal administration which contain the active ingredient SLPI or SLPI-expressing microorganisms, and also methods of producing these pharmaceutical compositions.

### BACKGROUND OF THE INVENTION

The chronic inflammatory intestinal diseases (IID), to which in the widest sense there belong enteritis necroticans, enteritis regionalis Crohn (Crohn's disease), colitis cystica, colitis granulomatosa, colitis gravis, colitis haemorrhagia, colitis ischaemica, colitis mucosa and colitis ulcerosa, are distinguished by phased, destructive inflammatory reactions of the intestinal mucosa. The most severe forms, Crohn's disease and colitis ulcerosa (ulcerative colitis), are differentiated in their distribution pattern and their macroscopic and histological picture.

Crohn's disease is an unspecific granulomatous inflammation which can affect all sections of the digestive tract from the esophagus to the anus, but above all is present in the region of the lower ileum and the colon. In about 40% of all cases the terminal ileum is exclusively affected, rarely the esophagus and stomach. In ulcerative colitis, a diffuse, continuous inflammation of the colonic mucosa is concerned, which is characterized by ulcerations with mucosal islands remaining between them; the disease encroaches on the small intestine only in rare cases. The definitive diagnosis of a chronic inflammatory intestinal disease can frequently succeed only through the chronic course. In ulcerative colitis, only the mucosa is affected, while Crohn's disease affects all wall layers and fistulas often form. However, a differentiation between Crohn's disease and ulcerative colitis is frequently impossible.

The reasons why a chronic inflammatory intestinal disease arises are extremely unclear. Thus Crohn's disease is attributed to immunological factors, genetic factors such as polygenic transmission, nutritional factors such as for example the frequent consumption of candy, and also infectious factors, for example rotaviruses, non-capsulated mycobacteria and pseudomonads. Likewise importance has been attached to the psychosocial environment and the premorbid personality structure. More recent research indicates that a pathologically increased activation of the mucosal immune system is probably of decisive importance for the pathogenesis of the chronic inflammatory intestinal diseases.

Since a causal therapy is not yet possible, the treatment of Crohn's disease and ulcerative colitis is principally aimed at alleviation of the symptoms. The established therapies for chronic inflammatory intestinal diseases are at present essentially based on unspecific, inflammation inhibiting substances such as glucocorticoids and aminosalicylates.

Glucocorticoids, by means of a reduction of the nuclear factor Kappa B, inhibit the synthesis of nearly all proinflammatory cytokines, the expression of adhesion molecules, and the production of prostaglandins and leucotrienes. A long-term prophylaxis with glucocorticoids is however not reasonable, since it has been shown that long term administration is accompanied by serious undesired effects. Abscesses are absolute contraindications for glucocorticoids; conglomerate tumors or intra-abdominal resistances and enteroenteral fistulas are relative contraindications. It has been shown that with newly developed glucocorticoids, for example budesonide, the side effects of the steroid therapy can be reduced, at least for a short time. In the acute phase, though, budesonide has to be so highly dosed that besides the topical effect a systemic effect is to be observed, even if comparatively small.

Aminosalicylates likewise decrease the nuclear factor kappa B and thereby the formation of pro-inflammatory cytokines or their receptors. This effect is however far more weakly manifested than in steroid treatment. Aminosalicylates are less effective overall than glucocorticoids in the treatment of chronic inflammatory intestinal diseases. The galenical formulations at present used were conceived with the aim of a different release characteristic, that is, a release from the proximal small intestine as far as the proximal colon. However, it has not been established up to now that the different anatomical release locations actually have a therapeutic advantage in the sense of a locally targeted therapy.

In certain clinical situations, the administration of glucocorticoids or aminosalicylates is accompanied by a change of nutrition. With severe phases of Crohn's diseases and ulcerative colitis, complete parenteral nutrition is absolutely necessary. Above all for children with growth disturbances or with severe steroid effects, a balanced enteral diet is prescribed. It has however been shown that there is no certainly effective diet for Crohn's diseases and colitis, since investigations on exclusion diet, reduced carbohydrate diet or fish oil preparations gave partially contradictory results (Stange and Schreiber, Deutsches Ärzteblatt, 22 (1997), 1493-1498).

In chronically active patients, an immunosuppressive therapy in the narrow sense, that is, with medicaments such as azathioprine, its metabolite 6-mercaptopurine, methotrexate and cyclosporine are used.

A positive effect of azathioprine and its metabolite 6-mercaptopurine on the healing and isolation of fistulas has repeatedly been described, but has not been substantiated in a prospective, controlled manner in large trials (Present et al., N. Engl. J. Med., 302 (1980), 981-987; Present et al., Annals Internal Medicine, 111 (1989), 641-649). It is also found to be disadvantageous that the average latency up to a therapeutic effect is about three months, and in any case about 20% of the patients need four to seven months to respond to the therapy. In ulcerative colitis, only a few studies have been performed on the use of azathioprine. However, they showed that the medicament is not indicated in acute ulcerative colitis. Azathioprine causes a series of side effects, including dose-independent allergic reactions such as nausea, diarrhea, joint pains and increase of liver enzymes, and dose-dependent side effects, such as cytopenia, infections, and toxic hepatitis.

Methotraxate is an immunosuppressive substance, which inhibits the enzyme dihydrofolate reductase and in this manner intervenes in purine metabolism. Methotrexate has numerous effects on the human immune system. It suppresses antibody production of B-cells, monocyte activation, neovascularization, and the activation of granulocytes. The use of methotrexate only takes place at this time when Crohn's disease has an azathioprine resistant course, however not in ulcerative colitis.

Cyclosporin A acts preferably on lymphocytes and inhibits their clonal expansion and proliferation. The clinical use of cyclosporin A in the treatment of chronically active Crohn's disease has been found to be effective in three out of four studies (Neurath and Stange, Deutsches Ärzteblatt, 28-29 (2000), 1672-1678). Moreover cyclosporine A frequently causes side effects such as hypertonia, diabetic metabolic aspects, renal insufficiency and occasional opportunistic infections.

Since 1980, approaches to selective immunomodulation have been developed, in order to treat chronically relapsing, unspecific intestinal inflammations. These are based on influencing the equilibrium between inflammation stimulating and inflammation inhibiting cytokines in the intestine. This is to be attained in that the secretion of pro-inflammatory cytokines is suppressed, or in that anti-inflammatory cytokines, such as interleukin-10 (IL-10) or interleukin 4 are stimulated or substituted in their formation.

In animal experiments, inactivation of the IL-10 gene led to the occurrence of chronic intestinal disease. Furthermore, recombinant IL-10 exerted a preventive effect on the development of an experimental colitis, the steroid amount required for therapy being reduced. In several studies, the potential therapeutic effect of IL-10 was evaluated in patients with Crohn's disease. According to clinical and endoscopic criteria, a remission was observed in about 30% of the patients. Similar results were also obtained with the use of IL-11 (Neurath and Stange, Deutsches Ärzteblatt, 97 (2000), 1672-1678). Steidler et al. (Science, 289 (2000), 1352-1355) describe the use of an IL-10-secreting recombinant Lactococcus lactis strain for the treatment of chronic inflammatory intestinal diseases in a mouse model. Living recombinant L. lactis bacteria were administered daily over a period of 14 days to diseased animals. It was found that by means of this method the same effect was attained as with the systemic administration of IL-10 or dexamethasone. L. lactis is a gram-positive, non-pathogenic bacterium which does not belong to the natural intestinal flora.

With the use of anti-inflammatory cytokines, for example interleukin-4, there is however likewise found a considerable potential for side effects. In a study by van Dullemen et al. (Gastroenterology, 109 (1995), 129-135) it was indeed found that with the single application of a humanized anti-TNF-α-antibody in patients with Crohn's disease, healing lasting for at least a few weeks was attained in 8-10 patients; however, strong side effects were observed. For example, antibody formation took place against this hybrid mouse/human antibody, which was accompanied by serum disease and occasionally by the development of lymphomas. It was likewise found that the irreversible reduction of cell populations effected by the antibody showed a considerable immunological risk, particularly with multiple administrations.

There are also numerous indications that infections contribute to the origination of the inflammatory intestinal disease formation cycle. Thus for example lymphocytes which were treated with an E. coli liposaccharide extract showed cytotoxic activity against epithelial cell colonies (Shorter et al., Gastroenterology, 58 (1970), 692-698). Patients with ulcerative colitis furthermore more frequently have hemolytic, enterotoxic or necrotoxic E. coli strains than do healthy subjects. A strategy for the treatment of, for example, ulcerative colitis therefore consists of the administration of wide-spectrum antibiotics. However, no therapeutic action was found with vancomycin. Tobramycin, whose activity is mainly directed against gram-negative bacteria such as E. coli, seems, though, to have short-term curative effects in ulcerative colitis.

Trials were also undertaken to effect long-term changes in the intestinal flora of chronic inflammatory intestinal disease patients. For example, ulcerative colitis patients were pre-treated with gentamycin and then treated with the non-pathogenic E. coli strain (Nissle 1917) (Mutaflor) (Rembacken et al., The Lancet, 354 (1999), 635-639). It was found that the E. coli strain exerted an effect similar to that of mesazaline (5-aminosalicylic acid), remission and period of remission being comparable.

Natural or recombinant bacterial strains were also used for the treatment of other diseases of humans and animals. Thus WO 99/26642 describes the use of the non-pathogenic E. coli strain DSM 6601 for the treatment of diarrhea in the veterinary field. Vandenplas (Clin. Mikrobiol. and Infect., 5 (1999), 299-307) describes the use of biotherapeutic means, particularly living bacteria and yeast cells, for the treatment of acute and chronic infective gastroenteritis. Paton et al. (Nature Medicine, 6 (2000), 265-270) describe the use of recombinant bacteria, for example recombinant Escherichia coli strains, which form a Shiga toxin receptor on their cell surface, for the treatment of gastrointestinal diseases which are caused by Shiga toxin producing bacteria. Beninati et al. (Nature Biotechnology, 18 (2000), 1060-1064) describe the use of two recombinant Streptococcus gordonii strains which secrete a microbiocide and can stably colonize the rat vagina, for the treatment of an experimentally produced vaginitis caused by Candida albicans.

It is known of a few endogenous proteolytic enzymes that they take part directly or indirectly in the pathogenesis of various diseases of the human or animal body. Endogenous proteolytic enzymes principally act to destroy invading microorganisms, antigen-antibody complexes, and certain tissue proteins which are no longer required by the organism. In a normal healthy organism, proteolytic enzymes are produced in a limited amount and are regulated by the synthesis of a series of protease inhibitors. Tissues which are particularly exposed to proteolytic attacks and infections, for example tissues of the respiratory organs, normally contain very many protease inhibitors. In certain cases, for example severe pathological processes such as sepsis or acute leukemia, the amount of free proteolytic enzymes is increased. A disturbance of the equilibrium between proteases and protease inhibitors can lead to severe damage to the organism concerned, in that, for example, protease-mediated tissue destruction occurs, to which belong emphysema, arthritis, glomerulonephritis, periodontitis, muscular dystrophy, tumor invasion and other pathological states.

The secretory leucocyte protease inhibitor (SLPI), which inhibits enzymes with serine protease activity, belongs to the protease inhibitors identified up to now. The 12-kilodalton protein is above all detected in such locations in the body where this is in direct contact with its environment, for example in the parotid gland and in the epithelia of the nasal cavity, the trachea, and the bronchi. SLPI inhibits among other things human leucocyte elastase, cathepsin G and human trypsin. Leucocyte elastase is a serine protease of particular interest, since the enzyme when released extracellularly destroys connective tissue and proteins associated therewith. Leucocyte elastase has been connected with different pathological states, for example emphysema and rheumatoid arthritis. Trypsin is likewise a protein of particular interest, since it is known that trypsin can initiate the destruction of certain internal organ tissues, for example pancreatic tissue during pancreatitis. Capthepsin G is known to be capable of destroying a series of proteins in vitro, for example, proteins of the complement metabolic pathway. SLPI furthermore has antiviral, antimycotic, and antibacterial effects.

SLPI also appears to play a part in the origin of chronic gastritis. Thus Nilius et al. (in: Cellular Peptidases in Immune Functions and Diseases 2 (ed.: Langner and Ansorge), (2000), 445-454. (Kluwer Academic/Plenum Publishers) show that in a Helicobacter pylori infection of the gastric mucosa, the SLPI formed and secreted by the epithelial cells of the gastric mucosa is significantly reduced.

The use of SLPI for the therapy of various diseases is known.

Thus U.S. Pat. No. 5,633,227 discloses a method for the treatment of disease states mediated by mast cells in mammals by the administration of a pharmacologically effective SLPI fragment or a mutein thereof. Likewise a method for the treatment of asthma or allergic rhinitis by the use of SLPI is described. The publication also discloses a method of inhibiting tryptase or tryptase-mediated disease states by the administration of SLPI peptides or protein portions.

U.S. Pat. No. 5,851,983 discloses a polypeptide which includes the C-terminal portion of SLPI and thus can inhibit elastase. Likewise there are described a pharmaceutical composition containing this polypeptide and a method of treating diseases which a rise from excessive activation of neutrophils, or which are connected to neutrophil protease. For example, inflammatory diseases, thrombocyte aggregation thromboses, and reperfusion damage after ischemia can be treated, and also diseases such as chronic bronchitis, ARDS [Acute Respiratory Distress Syndrome], renal inflammation, pulmonary inflammation, etc.

WO 94/06454 describes a method of inhibiting retrovirus infections, particularly infections with HIV, SLPI proteins or derivatives thereof being administered. The publication furthermore discloses specific SLPI-coding nucleotide sequences, and the proteins coded for by these sequences.

WO 99/17800 discloses a pharmaceutical composition including SLPI protein. This medicament is particularly designed for the treatment of respiratory diseases, for example pulmonary diseases, for the treatment of diseases which are characterized by elevated protease levels, and for the treatment of diseases mediated by leucocytes or mast cells.

U.S. Pat. No. 6,132,990 discloses methods for the production of recombinant serine protease inhibitors and DNA sequences used therefore. The disclosed protein can inhibit chymotrypsin and elastase, but not trypsin.

JP 07-103977 A describes a method for the detection of SLPI and SLPI-elastase complexes using antibodies directed against SLPI. The system is used in particular for the detection of respiratory infections.

However, there are hardly any investigations relating to the occurrence and function of SLPI in the intestine, and the results are in part very contradictory. Thus Bergenfeldt et al., J. Gastroenterol., 31 (1996), 18-23, describe an immune staining for SLPI in epithelial cells of the human intestinal mucosa. Si-Taher et al., Gastroenterology 118 (2000), 1061-1071, describe a constitutive and regulated secretion of SLPI in human intestinal epithelium, and they also show an antibacterial activity of SLPI against the pathogen Salmonella typhimurium. Franken et al., J. Histochem. Cytochem., 37 (1989), 493-498, however report that SLPI is not, or hardly, present in the digestive tract. In an investigation by Nystrom et al., Scand. J. Clin. Lab., Invest., 57(2) (1997), 119-125, the question was researched of to what extent the SLPI derived from saliva and swallowed can contribute to the amount of SLPI found beforehand in the intestine. The authors come to the conclusion that swallowed SLPI is quickly destroyed in the stomach and duodenum, and consequently plays no part for inflammatory diseases in the intestinal tract.

### BRIEF SUMMARY OF THE INVENTION

The present invention thus has as its object the technical problem to provide means which can be used for the treatment of chronic inflammatory intestinal diseases, and methods for the production and use of such means, where the means are to make possible to a grater extent than the means known up to now a treatment of the causes of the chronic inflammatory intestinal diseases and, in contrast to the means used up to now, to make possible a topical therapy, without the appearance of the systemic side effects described in the prior art.

The present invention solves this technical problem, particularly by the use of an effective material, selected from the group consisting of secretory leucocyte protease inhibitor (SLPI), a fragment thereof, a complex thereof, a derivative thereof, an analog thereof, an expressible nucleic acid coding for the effective material SLPI or a fragment or derivative thereof, and a non-pathogenic microorganism containing the nucleic acid and capable of SLPI formation, for the treatment of a disease of a human or animal body, selected from the group of enteritis necroticans, enteritis regionalis Crohn (Crohn's disease), colitis cystica, colitis granulomatosa, colitis gravis, colitis haemorrhagia, colitis ischaemica, colitis mucosa and colitis ulcerosa (ulcerative colitis).

The ulcers with deep fissures which appear particularly in Crohn's disease indicate that a proteolytic destruction of the intestinal tissue takes place in the chronic inflammatory intestinal diseases. The intestine is in general characterized in that a rapid turnover of material takes place at the surfaces. The destruction and the production again of the extracellular matrix must therefore be based in healthy tissue on a close-meshed control in order to prevent erosion and ulcer formation and consequent impairment of the intestinal function. It was now surprisingly established according to the invention by immunological staining that the amount of secretory leucocyte protease inhibitor in the intestinal mucosa of Crohn's disease patients is drastically reduced in comparison with the intestinal mucosa of healthy patients. This surprising finding shows that in the intestinal epithelial cells of chronic inflammatory intestinal disease patients the equilibrium between serine proteases with proteolytic action and the protease inhibitor SLPI is disturbed. SLPI therefore cannot exert its function of protecting the epithelial tissue, as for example evidenced in the respiratory tract, so that proteolytic enzymes can destroy the intestinal epithelial layers. By the directed supply of SLPI into the organs concerned, it is thus possible to re-establish the equilibrium between the protease inhibitor and the inflammatory proteases in intestinal epithelial cells of Crohn's disease and ulcerative colitis patients. As such inflammatory proteases, neutrophil elastase, cathepsin G, and chymasen can be concerned, which in particular are derived from the neutrophil and eosinophil granulocytes and macrophages which arise to an amplified extent in the intestinal mucosa of chronic inflammatory intestinal disease patients.

### BRIEF DESCRIPTION OF THE DRAWINGS

The invention is described in detail by means of the following Figures and the example.

FIG. 1 shows the immune staining of a histological intestine section of a healthy patient, using a rabbit antibody specifically directed against human SLPI.

FIG. 2 shows the immune staining of a histological intestine section of a chronic inflammatory intestinal disease patient, using a rabbit antibody specifically directed against human SLPI.

### DETAILED DESCRIPTION OF THE INVENTION

In a particularly preferred embodiment of the invention, it is thus provided that the effective material, i.e., SLPI, a fragment thereof, a complex thereof, a derivative thereof or an analog thereof is used for the treatment of chronic inflammatory intestinal diseases, in that the effective material itself, preferably in isolated and purified form, is supplied to the organs concerned. The directed supply of the effective material, for example SLPI itself, to the anatomical regions concerned in chronic inflammatory intestinal disease patients effects a protection of the intestinal surface from destruction by the proteolytic activity of proteases. Since SLPI is furthermore known to have antiretroviral, antimycotic and antibacterial effects, the directed supply of SLPI into the intestine furthermore leads to combating secondary infections which frequently accompany chronic inflammatory intestinal diseases. To these belong, for example, infections by salmonellas and by enterotoxigenic coli bacteria.

In a further particularly preferred embodiment of the invention, it is provided that not the isolated and purified effective material itself, but an expressible nucleic acid which codes for the effective material SLPI or a fragment or derivative thereof is contained in a living, non-pathogenic microorganism capable of SLPI formation, is used for the treatment of diseases of the chronic inflammatory intestinal disease group. Here the non-pathogenic microorganism which contains nucleic acid coding for the effective material, for example SLPI and expresses the effective material, is infiltrated into the intestine, where it preferably inhabits the intestine and then expresses the effective material within the intestinal lumen over a given, preferably long, period and directly releases it to the cells of the diseased intestinal epithelium. In this manner, the same advantageous effects on diseased intestinal regions are attained as with treatment with the isolated and purified effective material itself. A directed, topical therapy of chronic inflammatory intestinal diseases is thus possible. In contrast with treatment with isolated and purified effective material, the treatment with a living, SLPI-producing microorganism is substantially more cost-effective. Furthermore, the dose required for treatment is also considerably reduced, so that the potential for side effects is likewise reduced.

This embodiment furthermore offers several additional advantages. If an Escherichia coli strain is concerned as the non-pathogenic microorganism used, for example the E. coli strain (Nissle 1917), the advantageous effect of SLPI can be combined with the favorable effect of E. coli (Nissle 1917) on the remission of chronic inflammatory intestinal diseases, as described in the prior art. Since a given amount of the effective material SLPI is given up directly to the tissues concerned continuously and over a long period by the microorganisms, for example bacterial, concerned, the bioavailability of the effective material SLPI is extraordinarily high, since pharmaceutical factors, such as production methods, solubility, etc., which influence the bioavailability of an effective material with conventional medicaments, play no part. Also the pre-systemic elimination (first pass effect), that is, the metabolism of the effective material SLPI, which otherwise considerably limits the bioavailability of effective materials, plays only a subordinate part. An advantage not to be underestimated furthermore consists in that the cost-intensive isolation and purification of the effective material SLPI from bacteria or animal or human tissues do not apply.

In connection with the present invention, under the concept “chronic inflammatory intestinal diseases” there are understood chronic/relapsing specific inflammations of the intestine, particularly ulcerative colitis and Crohn's disease. The concept likewise includes all diseases which fall under the heading of “indeterminate colitis” and in which no clear allocation to a given clinical picture is possible. The concept likewise includes all extra-intestinal diseases accompanying chronic inflammatory intestinal diseases, for example, chronic hepatitis, cirrhosis, granulomatosis, urolithiasis, amyloiodosis, erythema nodosum, pyoderma gangrenosum, stomatitis aphthosa, arthritis, tympanitis, uveitis/iritis, autoimmune hemolytic anemia, vasculitis, fibrous alveolitis, pericarditis, hyperthyroidism, and the like.

In connection with the present invention, by “effective material” is understood SLPI itself, fragments thereof, complexes thereof, derivatives thereof or analogs thereof, as long as these have the biological activity required for use according to the invention. The concept SLPI in general is used hereinafter with the same meaning as the said concept. In connection with the present invention, under the concept “effective material” are also understood medicaments which can be used either prophylactically or accompanying a disease, in order to prevent, alleviate or eliminate disease states.

By “secretory leucocyte protease inhibitor (SLPI)” is understood according to the invention a eukaryotic protein which exerts an inhibitory effect on serine proteases, particularly leucocyte elastase, trypsin and cathepsin G, and which furthermore possesses antiretroviral, antimycotic and antibacterial activity. The effective material SLPI used according to the invention can be of natural origin, for example, a protein isolated from a eukaryotic tissue, preferably a mammalian tissue, preferably from a human tissue. The effective material SLPI can also be a protein produced by means of DNA recombination techniques or of synthetic origin, for example, a protein produced by use of the solid phase synthesis method of Merrifield (Angew. Chem., 97 (1985), 801).

In connection with the present invention, by “fragments” are understood portions of the SLPI protein which are of sufficient length to be able to exert the said activities. Thus according to the invention, by a fragment of SLPI is understood a protein portion which has fewer amino acids than native SLPI, that is, fewer than 132 amino acids, but in which the two main domains, namely the carboxy-terminal region which has the antiproteinase activity, and the amino-terminal region, which exerts the antimicrobial effect against, for example, Staphylococcus aureus, are retained. Preferably such a fragment is characterized by the presence of four disulfide bridges, so that the tertiary structure of the protein substantially remains maintained.

According to the invention, by “complex” is understood a compound which besides SLPI includes several other components, for example, a multi-enzyme complex or a heteromeric protein which consists of an ordered association of functionally and structurally different enzymes including SLPI, for example an SLPI-elastase-1 complex. According to the invention, an SLPI complex can be a natural SLPI complex. A SLPI complex produced in vitro can also be concerned, however, which includes other protease inhibitors, for example, α₁-macroglobulin, α₁-protease inhibitor (α₁-PI), α₁-antichymotrypsin, α₁-anticollagenase and α₁-trypsin inhibitor.

In connection with the present invention, by “derivatives” are understood functional equivalents or derivatives of SLPI which are obtained with retention of the basic SLPI structure by substitution or atoms or molecular groups or residues and/or with their amino acid sequences differing from the naturally occurring human or animal SLPI proteins at least one position, but which substantially have a high degree of homology on the amino acid level and comparable biological activity. According to the invention, the concept “derivative ” also includes fusion proteins, in which functional domains of another protein, for example, another protease inhibitor, are present in the N-terminal portion or in the C-terminal portion. “Homology” means in particular a sequence identity of at least 80% and particularly preferred, at least more than 90%, 95%, 97% and 99%. The expression “homology,” known to one skilled in the art, thus denotes the degree of relationship between two or more polypeptide molecules which is determined by the agreement between the sequences. Here an agreement can mean both an identical agreement and also a conservative amino acid exchange.

The differences between a derivative and native SLPI can for example arise by mutations, such as for example deletions, substitutions, insertions, displacements, base exchanges and/or recombinations of the nucleic acid sequence coding for the amino acid sequence. Of course naturally occurring sequence variations are also concerned here, for example, sequences from another organism or sequences which were naturally mutated, or mutations introduced in a directed manner, by means of the usual means known to one skilled in the art, for example, chemical agents and/or physical agents, into the corresponding sequences.

In connection with the present invention, by “nucleic acid coding for SLPI or a fragment or derivative thereof” is understood a nucleic acid which codes for a SLPI protein, fragment or derivative thereof, which has the functional domains, particularly the antiproteinase activity, the antiretroviral activity, the antimicrobial activity and the antimycotic activity, of native SLPI. The nucleic acid sequence used according to the invention can be a DNA or RNA sequence in linear or circular form. The nucleic acid can be a nucleic acid isolated from natural sources, for example, from eukaryotic tissues, preferably from mammalian tissues, more preferably from human tissues, or can be synthetically produced.

Since the SLPI sequences derive from a eukaryotic organism, preferably from a mammal, more preferably from humans, the sequence coding for SLPI used according to the invention, in the case of its use in a non-pathogenic bacterium, must have a form which ensures its expression in the bacterium, i.e., a prokaryotic microorganism. In the case that the sequence used according to the invention thus has to be expressible in a prokaryotic organism, a nucleic acid isolated from natural sources is preferably modified so that, for example, its intron sequence is removed, since most bacteria do not have available any suitable cellular mechanisms for the correct removal of the intron sequence. In this case, the native sequences of the nucleic acid coding for a signal peptide are also removed, since the proteins of bacteria have, if at all, signal sequences other than those of eukaryotes. If necessary, the codon composition of the nucleic acid derived from an eukaryotic tissue is also modified in dependence on the host organism, in order to attain a more efficient expression of the eukaryotic gene in the prokaryotic host organism. It is known that prokaryotes have a tRNA population different from that of eukaryotes and therefore frequently use other codons. This different codon usage can limit an efficient expression of eukaryotic genes in bacteria.

If the sequence coding for SLPI and used according to the invention is to be used in non-pathogenic fungal microorganisms, for example, ascospore-forming yeasts such as Saccaromyces boulardii, their naturally present intron sequences have to be removed if necessary. Yeast cells admittedly possess cellular mechanisms for removing intron sequences, but there are differences from higher eukaryotes. If necessary, the native signal peptide coding sequences of the sequence used according to the invention also have to be removed, since it has been found that a few, but not all, signal sequences of mammalian proteins are recognized and correctly processed by the yeast cell. A modification of the codon composition in the sequence used according to the invention is however not required, since in yeast cells high expression rates are observed of foreign genes, particularly eukaryotic genes.

The expression “a non-pathogenic microorganism capable of forming SLPI” in connection with the present invention means that a microorganism used according to the invention does not have pathogenic effects on the macroorganisms, i.e., humans or animals, into which it is to be infiltrated, and that it can correctly transcribe and translate the nucleic acid derived from a eukaryotic organism and if necessary brought into an expressible form, a protein with the activity of SLPI being produced in the cytoplasm of the microorganism and transported out of the cytoplasm through the outer membranes into the periplasmic space, and preferably released to the environment of the microorganism. Thus it is provided according to the invention that a microorganism infiltrated into the intestinal sections concerned of chronic inflammatory intestinal disease patients is able to express a protein with SLPI activity over a given period, and directly release it to the intestinal epithelial tissue. Preferably the non-pathogenic microorganism is thus capable of living for a given period in the intestine of a human or animal and possibly of colonizing this. The observed SLPI deficiency in the intestinal mucosa of chronic inflammatory intestinal disease patients can be compensated in this way, and the clinical manifestations connected therewith can be eliminated.

In a preferred embodiment of the present invention, the use of the effective material SLPI for the treatment of chronic inflammatory intestinal diseases takes place in that the effective material, preferably isolated and purified, is administered in a pharmaceutical composition. In connection with the present invention, by “pharmaceutical composition” there is understood a mixture including a naturally or synthetically produced effective material which is used for diagnostic, therapeutic, and/or prophylactic purposes, the effective material being contained in a form well applicable in patients. The pharmaceutical composition can be a solid or liquid mixture. For example, a pharmaceutical composition including SLPI can contain one or more pharmaceutically acceptable excipients. The pharmaceutical composition can include further additives such as stabilizers, thickeners, parting agents, lubricants, colorants, odorous substances, taste substances, emulsifiers or similar materials known in the art.

It is particularly provided according to the invention that the isolated and purified effective material contained in a pharmaceutical composition is administered to a chronic inflammatory intestinal disease patient in a dose sufficient to heal or prevent the state of the chronic inflammatory intestinal disease, to stop the progression of the chronic inflammatory intestinal disease and/or to alleviate the symptoms of the chronic inflammatory intestinal disease. The dosage of the effective material therefore takes place so that an optimum therapeutic effect is attained without substantial toxic side effects, and the success of the treatment lasts for a long time.

It is particularly provided according to the invention that the isolated and purified effective material contained in the pharmaceutical composition is administered once through three times a day in a dose of 1-5000 mg of effective material. The amount of the effective material to be administered to a patient depends on, among other things, the form of administration, the age, sex and body weight of the patient to be treated, and on the severity of the disease. The exact dose with which a patient is to be treated must therefore be individually established by the treating doctor.

In a preferred embodiment of the present invention, it is provided that the isolated and purified effective material contained in the effective material is administered orally. An oral administration of the effective material is preferred particularly in such chronic inflammatory intestinal diseases which relate to the upper intestinal tract, such as the duodenum or small intestine, The effective material is preferably administered in the form of a suspension, tablet, pill, capsule, lollipop, granulate, powder, or similar administration form. Although it has been shown that SLPI is relatively stable to acids (Nystrom et 1., Scand. J. Clin. Lab . Invest., 57 (1997), 119-125), forms of medicament are preferred which have a coating resistant to gastric juice, so that the effective material can pass through the stomach unhindered and preferably first goes into solution in the upper intestinal sections. The composition of coatings resistant to gastric juice and methods for their preparation are known in the art. In particular, medicaments to be administered orally are preferred which have a delayed release mechanism for the effective material, in order to provide topical long-term therapy from the lumen to the intestinal mucosa of chronic inflammatory intestinal disease patients. The construction and composition of such medicament forms with delayed release of effective material are likewise known in the art.

In another embodiment of the invention, it is provided to rectally administer a pharmaceutical composition containing the isolated and purified effective material. A rectal administration of the effective material is preferred in the treatment of chronic inflammatory intestinal diseases which in particular concern the lower intestinal region. For example in ulcerative colitis which always begins in the rectum and propagates in the proximal direction in many affected persons. The administration of the effective material preferably takes place in the form of a suppository, enema, foam or similar administration form.

It is provided in a further embodiment of the present invention that the preferably isolated and purified effective material is administered parenterally, that is, bypassing the gastrointestinal tract. A parenteral administration of the effective material can be indicated in particular when the therapy of the chronic inflammatory intestinal disease is accompanied by parenteral nutrition. This form of therapy can furthermore be advantageous in children with growth disorders. It is provided according to the invention that parenteral administration of the effective material particularly takes place by injections or infusions.

In a particularly preferred embodiment of the invention, the treatment of a chronic inflammatory intestinal disease patient takes place, not with the isolated and purified effective material SLPI itself, but with a non-pathogenic microorganism capable for forming SLPI and containing an expressible nucleic acid coding for the effective material SLPI or a fragment or derivative thereof. It is particularly provided according to the invention that the non-pathogenic microorganism is capable of producing the effective material before, during or after administration to a human or to an animal, and to release the produced effective material to the diseased orgasm of the digestive tract after the administration.

In a particularly preferred embodiment of the invention, it is provided that the concerned non-pathogenic microorganisms are bacterial or fungal microorganisms which belong to the commensals of humans or animals. In connection with the present invention, “commensals” are understood as non-pathogenic microorganisms which live from the nutrition of their host, for example a human or animal, respectively its secretions, for example saliva or mucus. Such commensals live on, among other things, the mucosa of the mouth, of the respiratory, urinary and sexual organs, or in the intestine. With commensals used according to the invention, saprophytic microorganisms are preferred, but not commensal microorganisms living parasitically, which are often pathogenic.

In a particularly preferred embodiment of the invention, it is provided that a fungal, non-pathogenic, commensal microorganism is used as the host cell for a nucleic acid coding for the effective material SLPI or a fragment or derivative thereof. As host organisms for the expression of eukaryotic foreign genes, the fungal microorganisms belonging to the eukaryotes, for example yeasts, possess a few decisive advantages over the bacteria, belonging to the prokaryotes. For example, yeast cells can secrete the gene products of eukaryotic genes, that is, transport the gene products out of the cell and release them to the environment. The proteins can be glycosylated during secretion. Very large gene fragments can also be cloned in yeast cells. Yeast cells are therefore particularly suitable for cloning and expression of SLPI and its release to the intestinal epithelium.

The fungal microorganism preferably belongs to the genus Saccharomyces, that is, to the ascospore-forming yeasts. In a particularly preferred embodiment, the fungal, non-pathogenic, commensal microorganism used according to the invention is Saccharomyces boulardii.

In a particularly preferred embodiment of the invention, it is provided that the non-pathogenic microorganisms belong to the natural intestinal flora of humans or animals. This is particularly advantageous insofar as the patient's intestinal flora is not infiltrated with germs whose influence on the composition of the natural intestinal flora, or respectively the pathological events connected with chronic inflammatory intestinal diseases are unknown or difficult to evaluate. A particular advantage furthermore consists in that the microorganisms used according to the invention are physiologically very well adapted to the special conditions within the mammalian intestine, so that the microorganisms used according to the invention can successfully compete for nutrition with the germs within the patient's intestine. Thus a long-term persistence of the microorganisms used according to the invention, and a resulting long-term expression of the effective material SLPI, are ensured. Furthermore, microorganisms of the normal intestinal flora mediate a protective infection against pathogenic or opportunistic microorganisms.

In a particularly preferred embodiment of the invention, it is provided that the non-pathogenic microorganisms used are an aerobic or anaerobic gram-negative bacterium of the human or animal intestinal flora. The gram-negative host bacterium used preferably belongs to the genera Escherichia, Pseudomonas, Bacteroides, or Proteus.

In a particularly preferred embodiment of the invention, the gram-negative host bacteria used are of the strain Escherichia coli (Nissle 1917), which corresponds to Escherichia coli DSM 6601. This strain is non-pathogenic for humans. It is known for E. coli Nissle 1917 (serotype 06:K5:H) that this strain shows antagonistic activity against different pathogenic and non-pathogenic enterobacteria. The antagonistic activity of E. coli (Nissle 1917) is probably to be attributed to the production of bacteriocins or microcins (Blum, Marre and Hacker, Infection, 23 (1995), 234-236), but can also be connected with the blocking of receptors of the intestinal mucosa (Rembacken et al., The Lancet, 354 (1999), 635-639). It is furthermore known for E. coli (N isle 1917) that ulcerative colitis patients treated with this strain showed remissions which were comparable with those of the medicament mesalazin, without however the side effects known for mesalazin arising (Rembacken et al., The Lancet, 354 (1999), 635-639). The strain E. coli (N isle 1917) thus offers the particular advantage that the favorable effect of the wild type strain on the course of chronic inflammatory intestinal diseases can be combined with the advantageous effect according to the invention of a SLPI supply to the healing process. E. coli (N isle 1917) is commercially obtainable under the name “Mutaflor ” from Ardeypharm GmbH , Herdecke, Germany. Escherichia coli furthermore offers the great advantage it is the best researched microorganism, which is the most frequently used in experiments in gene technology. Very many gene-technical methods and cloning vectors are known for this bacterium.

In a further preferred embodiment of the invention, it is provided that the non-pathogenic microorganism used is an aerobic or anaerobic, gram-positive bacterium of the natural intestinal flora. It is known that the normal intestinal flora is populated by many gram-positive bacteria, among which are, for example, kinds of Bifidobacterium, Streptococcus, Staphylococcus and Bifidobacterium. For example, Bifidobacterium bifidum is the predominant intestinal species in breast-fed babies, but is also a substantial proportion of the normal intestinal flora of bottle-fed children and of adults, and possibly all warm-blooded animals. Gram-positive bacteria have the decisive advantage over gram-negative bacteria as host organisms for the expression of eukaryotic genes that they can secrete the gene products of eukaryotic genes, that is, can transport the gene products out of the cell and release them to the environment. Gram-positive host bacteria are therefore particularly suitable for the expression of SLPI and release to the intestinal epithelium.

A preferred embodiment of the present invention therefore includes the use of gram-positive bacteria of the genera Bifidobacterium, Streptococcus, Staphylococcus and Corynebacterium as host bacteria for the effective material SLPI or a fragment or derivative thereof. In a particularly preferred embodiment, the gram-positive host bacterium used is Streptococcus gordonii, which is a non-pathogenic and naturally transformable commensal bacterium (cf. Beninati et al., Nature Biotechnology, 18 (2000), 1060-1064).

In a further preferred embodiment of the invention, it is provided that for the expression of nucleic acids coding for the effective material SLPI, non-pathogenic microorganisms are used which do not belong to the natural intestinal flora or are not commensals of humans or animals, insofar as they are capable of forming SLPI and are non-pathogenic for the host into which they are to be introduced. Preferably bacteria are concerned as such microorganisms, and can live for at least a given period in the intestine of humans or animals. Such bacteria should furthermore have no disadvantageous effect on the course of a chronic inflammatory intestinal disease or on the therapeutic effect of SLPI. Preferred examples of bacteria which do not belong to the natural intestinal flora or are not commensals, but however can be used as host cells for an expressible nucleic acid coding for the effective material SLPI or a fragment or derivative thereof, include bacteria which are used for the fermentative production of foodstuffs. Particularly preferred examples are lactic bacteria, such as Lactococcus lactis, Lactobacillus delbrueckii subspec. bulgaricus, Lactobacillus casei,_{—}Lactobacillus caucasicus, Lactobacillus kefir, Streptococcus thermophilus, a few species of Leconostoc, and the like.

In a further preferred embodiment of the invention, mutants of the non-pathogenic microorganisms used according to the invention for expression of the nucleic acid coding for the effective material SLPI, in which the external cell integument is modified so that certain expressed proteins can leave the cell and reach the environment of the cell. Such mutants are also termed “leaky mutants”. Leaky mutants with modified cell integuments can be obtained by means of known methods, such as for example mutagenic methods using nitrosoguinidine. Examples of different types of leaky mutants are described by Anderson, Wilson and Oxender in J. Bacteriol., 140 (1979), 351-358, and Fung, MacAlister and Rothfield in J. Bacteriol., 133 (1978), 1467-1471. The use of leaky mutants as host cells for nucleic acids coding for the effective material SLPI thus have the advent age that release is ensured of the expressed SLPI protein to the environment, that is, to the intestinal epithelium of the chronic inflammatory intestinal disease patient.

In a further advantageous embodiment, spheroblasts, L-forms or protoblasts of gram-negative or gram-positive host bacteria or of fungal host cells are used. Bacterial spheroblasts are cells which are obtained by treatment of gram-negative bacteria with lysozyme. Bacterial protoblasts are cells which are obtained by treatment of gram-positive bacteria with lysozyme. Spheroblasts can also be recovered by means of a treatment with penicillin or lysozyme-EDTA. L-forms of gram-negative or gram-positive bacteria are characterized in that they have lost the capability of forming a functional cell wall. Methods of obtaining bacterial L-forms are described, for example, by Makemson and Darwish, Infect. Immunol., 6 (1972), 880. Spheroblasts can also be obtained from yeast cells by means of well-known methods.

According to the invention it is in particular provided that the nucleic acid coding for SLPI or a fragment or derivative there of and contained in the non-pathogenic microorganism is inserted in a vector. In connection with the present invention, the concept “vector” means an extrachromosomal DNA, in which is preferably concerned a plasmid, a cosmid, a bacteriophage, a virus, a shuttle vector and another vector usually used in gene techniques. The vectors according to the invention can have further functional units which effect, or at least contribute to a stabilization, selection and/or replication of the vector in a host organism.

It is particularly provided according to the invention that in contrast to the vectors usually used for the cloning of gene sequences, the vectors according to the invention for insertion of SLPI sequences contain no selection marker which rests on an antibiotic resistance. Since the host cells into which the vector for expression of the effective material is introduced are to stably populate the intestine of a chronic inflammatory intestinal disease patient for a given period, there would otherwise exist the risk that an antibiotic resistance contained in the vector would be passed on to other microorganisms of the intestinal flora and thus propagate within the intestinal flora.

It is therefore provided according to the invention that the selection marker contained on the vector in a preferred embodiment is a gene whose gene product is not damaging to the human or animal organism and which is easily detected. In a preferred embodiment, there is concerned as a selection marker contained on the vector a sequence coding for the green fluorescent protein (GFP), and the detection of the GFP product takes place, for example, by means of FACS or throughflow cytometry.

In a particularly preferred embodiment of the invention, a nucleic acid coding for the effective material SLPI or a fragment or derivative thereof is inserted into a vector so that it is under the functional control of at least one regulating element which ensures the transcription of the nucleic acid into a translatable RNA and/or the translation of the RNA into a protein, during or after the administration.

Regulating elements can for example be promoters, ribosome binding locations, signal sequences, and/or transcription terminal sequences. Regulating elements which are functionally connected to a nucleic acid coding for SLPI or a fragment or derivative thereof can be nucleotide sequences which are derived from other organisms or other genes than the nucleotide sequence coding for the SLPI.

According to the invention, as the promoter used, a constitutive or inducible promoter can be concerned. A promoter is the region of a DNA to which the enzyme RNA polymerase binds and initiates the process of gene transcription. A “constitutive promoter” is a non-regulatable promoter which, without external stimulus, continuously effects the transcription of a coded DNA sequence. An “inducible promoter” is a regulatable promoter which is activated directly by the presence or absence of a chemical means or indirectly by a stimulus from the environment such as a temperature change. A constitutive promoter has a disadvantage as against an inducible promoter insofar as an uncontrolled expression of a foreign protein, for example in a bacterial host cell, can lead to the dying out of this host cell.

According to the invention, the use of an inducible promoter or the expression of the nucleic acid coding for SLPI is therefore provided. In a preferred embodiment of the invention, an inducible promoter is used which is inducible by lack of nutrient. A promoter inducible by lack of nutrient is activated when the concentration of a chemical means which is necessary for the maintenance of cellular function, is strongly reduced or completely lacking. Such a promoter is in particular suitable for the specific growth conditions with which germs are confronted in the intestine. The nutrient supply of the germs undergoes most extreme fluctuations in the intestine. Thus when there is a lack of nutrient in the intestine, that is, when the intestine contains little or no chyme, the transcription of the nucleic acid coding for SLPI is induced by the inducible promoter used according to the invention. The SLPI protein then formed can diffuse, after infiltration out of the host cell used according to the invention, relatively unhindered to the intestinal epithelium, since the intestine contains little or no chyme.

It is particularly provided according to the invention that for expression of the nucleic acid coding for SLPI in a gram-negative host bacterium, for example E. coli ( Nissle 1917), an inducible promoter is used which is induced by a deficiency of phosphate. In a particularly preferred embodiment, the promoter used is the phoA promoter of Escherichia coli. In the case that the vector contains the phoA promoter, it preferably also includes the regulating genes phoB and phoR, in order to be able to switch the promoter on and off efficiently. In a further particularly preferred embodiment, the promoters used for effective material expression in a gram-negative host bacterium is the trp-, lac-, or tac- promoter of Escherichia coli. The promoters of E. coli can in principle also be used in gram-positive bacteria.

According to the invention it is particularly provided that for the expression of the nucleic acid coding for SLPI in a yeast cell, for example Saccharomyces boulardii, an inducible promoter is used which is induced by phosphate deficiency. In a particularly preferred embodiment, the promoter used is the promoter of the yeast gene PHO 5. In a further preferred embodiment, it is provided to use the promoter. which is induced by glucose deficiency, of the SDH 1 gene of yeast for expression in a yeast cell of the nucleic acid coding for SLPI.

In a preferred embodiment of the invention, it is provided that the nucleic acid coding for SLPI is set for expression in a bacterial host cell under the functional control of a ribosome binding site. In connection with the present invention, by the concept “ribosome binding site” is understood a sequence which is complementary to the 3′-end of the bacterial 16-rRNA and act s to bind ribosomes. Ribosome binding sites are normally located 3-12 bases before an initiation codon and usually include 3-9 bases. According to the invention it is particularly provided that for the ribosome binding site used, a Shine-Dalgarno sequence with the consensus sequence 5′-AAGGAGGU-3′ is used.

In a further preferred embodiment of the invention it is provided that the nucleic acid coding for SLPI is connected in a host cell according to the invention to a signal sequence suitable for the respective host, that is, with a bacterial or fungal signal sequence. A “signal sequence” is a sequence which codes for a signal peptide which effects the secretion of a protein from the cytoplasm of a microorganism into the periplasmic space or into the environment of the microorganism. The signal peptide is a short segment of about 15-30 amino acids, located at the N-terminal of secreted and exported proteins. The cellular machinery of the host cell for processing proteins recognizes the signal sequence so that the expressed protein is secreted through the cell membrane or through the membrane of a organelle, removed by a specific protease during the secretion process. Since SLPI is a protein normally secreted by an eukaryotic organism, the natural signal peptide of the native SLPI protein will be replaced, according to the invention, by a signal peptide suitable for the respective host cell, so that the transport out of the host cell into the periplasmic space or into the environment of the microorganism is ensured.

In a particularly preferred embodiment of the invention, it is in particular provided that for expression of the nucleic acid coding for SLPI in a gram-negative host bacterium, for example E. coli (Nissle 1917), the signal sequence of the β-lactamase gene of E. coli, or the signal sequence of the ompA gene of E. coli is used, in order to attain a secretion of the expresses SLPI protein into the periplasmic space and/or into the environment. According to the invention, it is also possible to use hybrid signal sequences, for example the sequence described by Konrad, Annals New York Academy of Sciences, 413 (1983), 12-22), which consists of a fusion of the first twelve amino acids of the β-lactamase signal sequence with the last 13 amino acids of the human insulin signal sequence.

In a further preferred embodiment of the invention, it is in particular provided that for expression of the nucleic acid coding for SLPI in a gram-positive host bacterium, for example, Streptococcus gordonii, the signal sequence of the a-amylase gene of Bacillus amyloliquefaciens or the signal sequence of the Streptococcus gene M6 is used in order to attain a secretion of the expressed SLPI protein through the cell wall into the environment.

In a further preferred embodiment of the invention, it is provided that for expression of the nucleic acid coding for SLPI in a fungal host cell, for example Saccharomyces boulardii, the signal sequence of the α factor of yeast or the signal sequence of the killer toxin of yeast are used in order to attain a secretion of the expressed SLPI protein through the cell wall into the environment.

In a particularly preferred embodiment of the present invention, it is provided that the living microbial host cell capable of SLPI expression and containing a nucleic acid coding for SLPI inserted in a vector, is administered in a pharmaceutical composition to a chronic inflammatory intestinal disease patient. According to the invention, it is in particular provided that the pharmaceutical composition contains sufficient colony-forming units (CFU) of the host cell capable of forming SLPI so that with multiple administration of the pharmaceutical composition according to the invention to a chronic inflammatory intestinal disease patient, the state of the chronic inflammatory intestinal disease is healed, the progression of the chronic inflammatory intestinal disease is stopped, and/or the symptoms of the chronic inflammatory intestinal diseases can be alleviated. According to the invention, it is in particular provided that a pharmaceutical composition contains 1×10⁸-1×10¹¹, preferably 1×10⁹-1×10¹⁰ CFU of the host cells according to the invention.

According to the invention, it is particularly provided that the pharmaceutical composition which contains the microorganism capable of SLPI formation is administered one to three times a day over a period of two to four weeks. The exact dosage depends on, among other things, the administration form, the age, sex and body weight of the patient to be treated, and the severity of the disease, and has to be individually established by the doctor.

For the pharmaceutical composition which according to the invention contains living microbial host cells, an oral administration form is concerned. The pharmaceutical composition to be administered orally can be administered orally, for example in the form of a suspension, tablet, pill, capsule, granulate or powder.

In a liquid pharmaceutical composition, the living microorganism according to the invention is present, free and not immobilized, in suspension. The suspension has a composition which ensures physiological conditions for a microorganism, so that in particular the osmotic pressure within the cell does not lead to lysis. A liquid pharmaceutical composition is above all suitable for microorganisms, particularly bacteria, with intact cell wall.

In a solid pharmaceutical composition, the microorganisms according to the invention can be present in free, preferably lyophilized form, or in immobilized form. For example, the microorganisms according to the invention can be enclosed in a gel matrix which provides protection for the cells. Inclusion in a gel matrix is particularly suitable for microorganisms whose outer membrane is partially or completely removed, and thus for leaky mutants, spheroblasts, protoblasts or L-forms. Such microbial forms are very fragile, and the inclusion in the gel matrix provides for the protection of the cells from mechanical shearing forces.

The microorganisms according to the invention, for example bacteria, can be included in a gel matrix in that a concentrated cell solution is mixed with a dissolved gelling medium and then the mixture is passed through needles of small diameter. Drops are thus formed which then fall into a solution which effects the gelling of the gelling medium and thus the formation of polymerized particles. Examples and modifications of this method are described in Brodelius and Mosback, Adv. Appl. Microbiol., 28 (1982), 1-25, and Klein, Stock and Vorlop, Eur. J. Appl. Microbiol. Biotechnol., 18 (1983), 86-91. Materials which can be used as the matrix for the inclusion of microorganisms include agar, alginates, carrageen, agarose or other polymers physiologically suitable for humans or animals and which can be gelled under physiological conditions.

Other forms of cell immobilization include adsorption of the microbial host cells according to the invention on solid supports or the immobilization of the host cells according to the invention by means of covalent bonds. These methods are described in Navarro and Durand, Eur. J. Appl. Microbiol. Biotechnol., 4 (1977), 243. An immobilization of bacteria according to the invention can also be attained in that the cells are enclosed between membranes whose pores are smaller than the bacteria themselves but large enough to make possible transport of the expressed SLPI proteins through the membrane. Such devices are well known and obtainable commercially (for example Amicon, Millipore, and Dorr-Olivier).

A solid pharmaceutical composition intended for oral administration and containing the host cells according to the invention in immobilized or non-immobilized form is preferably provided with a coating resistant to gastric juice. It is thereby ensured that the living microorganisms contained in the pharmaceutical composition can pass through the stomach unhindered and undamaged and the release of the microorganisms first takes place in the upper intestinal regions.

In a further preferred embodiment of the invention, the pharmaceutical composition containing the living host cells is administered rectally. A rectal administration preferably takes place in the form of a suppository, enema or foam. Rectal administration is particularly suit able for chronic inflammatory intestinal diseases which affect the lower intestinal sections, for example the colon.

The present invention therefore also concerns a pharmaceutical composition including at least one living cell of a non-pathogenic microorganism capable of SLPI formation, which contains an expressible nucleic acid coding for the effective material SLPI or a fragment or derivative thereof, the non-pathogenic microorganism preferably being a commensal or a component of the natural human or animal intestinal flora and/or usable for fermentative production of a foodstuff.

In a preferred embodiment of the invention, the pharmaceutical composition contains an anaerobic or aerobic, gram-negative or gram-positive bacterium of the natural human or animal intestinal flora. In a further preferred embodiment, for the microorganism contained in the pharmaceutical composition there is concerned a commensal yeast of humans or animals. In a further preferred embodiment of the invention, for the microorganism contained in the pharmaceutical composition there is concerned a bacterium which can be used for fermentative production of a foodstuff. In a further preferred embodiment of the invention, the pharmaceutical composition contains the pharmaceutical composition of a “leaky” mutant of a non-pathogenic microorganism.

In a particularly preferred embodiment of the invention, the pharmaceutical composition contains cells of the non-pathogenic Escherichia coli strain Nissle 1917. In a further particularly preferred embodiment of the invention, the pharmaceutical composition contains cells of the commensal bacterium Streptococcus gordonii. In yet another particularly preferred embodiment of the invention, the pharmaceutical composition contains cells of the commensal yeast Saccharomyces boulardii.

A particularly preferred embodiment relates to a pharmaceutical composition in which the microorganism contains a nucleic acid coding for the effective material SLPI or a fragment or derivative thereof, the nucleic acid being inserted into an expression vector and the expression of the nucleic acid being under the control of at least one regulating element, so that the effective material is expressed before, during or after administration of the pharmaceutical composition to a human or an animal, and after the administration, the pharmaceutical composition is released to the organs of the digestive tract.

The present invention therefore also relates to method for the production of a pharmaceutical composition, comprising:

(a) isolation or synthesis of a nucleic acid coding for the effective material SLPI;

(b) cloning of the nucleic acid coding for SLPI in a bacterial expression vector or a fungal expression vector;

(c) transformation of the recombinant expression vector obtained in (b) in a microbial host cell, where the host cell is a commensal of the human or animal intestinal flora and/or can be used for the fermentative production of foodstuffs;

(d) propagation of the transformed host cells;

(e) production of an immobilized, lyophilized, or liquid preparation of transformed host cells;

(f) mixing the immobilized, lyophilized, preparation or suspension of transformed host cells obtained in (e) with physiologically acceptable excipients, stabilizers, thickeners, parting agents, lubricants, emulsifiers or the like materials to obtain a pharmaceutical composition.

The isolation of a nucleic acid coding for the effective material SLPI can take place by means of methods usually used in gene technology (cf. Sambrook et al., Molecular Cloning: A Laboratory Manual, 2d. edition (1989), Cold Spring Harbor laboratory Press, NY, USA). Since the DNA sequence of the human SLPI gene is known (cf. U.S. Pat. No. 5,851,983 and U.S. Pat. No. 6,132,990), sequences coding for SLPI can for example be isolated and amplified from a eukaryotic tissue, preferably a mammalian tissue, most preferably a human tissue, with suitable primers using the method of the polymerase chain reaction (PCR). An amplification is particularly preferred with the use of a cDNA bank of a human tissue. Furthermore, the primer is preferably designed so that the coding SLPI sequence is provided at the 5′ and 3′ ends with suitable restriction interfaces. The amplification product is split with suitable restriction enzymes and after purification, for example using gel electrophoresis, and is cloned in a suitable vector.

In another embodiment of the invention, the sequence coding for SLPI can be produced synthetically. The chemical synthesis of the nucleic acid offers the advantage that the nucleic acid sequence can be modified with respect to the codon use, without changing the amino acid sequence of the coded protein. The synthesis of DNA sequences can for example take place using the phosphotriester method or the phosphate method, for example in solid phase systems. The synthesis preferably takes place using DNA synthesis devices, for example DNA automatic synthesis devices of Applied Biosystems. After purification of the synthesized sequence, this is inserted in a vector by suitable methods.

The insertion of the nucleic acid coding for SLPI, obtained either by amplification or by synthesis, into a suitable vector takes place using the usual methods used in the art, for example, restriction splitting and linking. A suitable vector must in general have the following properties:

(a) it must be able to integrate in a defined position and orientation the nucleic acid to be inserted;

(b) it must be able to penetrate with the integrated nucleic acid into a host organism, that is, be able to pass through the cell wall and cell membranes;

(c) it must behave in the host cell as a replicon, that is, as an independent genetic element; and

(d) the vector must be able to be duplicated when a cell divides, so that all progeny obtain at least one copy of t he vector.

Suitable vectors for gram-negative or gram-positive host cells or yeast cells are known in the art. As previously mentioned, the vector which is used according to the invention for cloning the nucleic acid coding for SLPI contains no selection markers which depend on an antibiotic resistance, but preferably contain a marker such as a gene sequence coding for the GFP protein, the gene product of which is easily detectable using FACS or throughflow cytometry. The vector also preferably already contains an expression cassette with a suitable promoter, a suitable ribosome binding site, a suitable signal sequence, and suitable transcription termination sequences.

After the insertion of the nucleic acid coding for SLPI into a suit able vector, the construct is introduced into a bacterial host organism or a yeast host organism. If a bacteriophage is concerned as the vector, this can be introduced by transduction into the host (cf. Sambrook et al., 1989). If a plasmid is concerned as the vector used, this can for example be infiltrated into the host by means of a transformation method. For Escherichia coli strains, the usual calcium transformation method is preferably used (cf. Sambrook et al., 1989). Transformation methods for Streptococcus cells are described, for example, in Clewell, Microbiol. Rev., 445 (1984), 409. Transformation methods for fungal host cells, for example yeast host cells, are likewise well known in the art.

After the transformation, transformed host cells are cultured and propagated in a suitable medium under suitable conditions until a suitable cell density is attained.

For the production of a suspension to be administered orally, the host cells are then suspended at a suitable cell density in a sterile physiological solution. The cultivated host cells can however also be lyophilized or immobilized using known methods. After lyophilization or immobilization, the cells, in a suitable CFU (colony forming unit) amount are mixed with materials such as pharmaceutically acceptable excipients, stabilizers., thickeners, parting agents, lubricants, colorants, odorous materials, taste materials, emulsifiers or the like pharmaceutically used materials, in order to produce a desired pharmaceutical composition.

The present invention relates, not only to the said uses of the effective material or of a microorganism capable of forming the effective material for the treatment of a previously defined disease, but also the use of a previously defined effective material, or a microorganism capable of forming this effective material, for the production of a pharmaceutical preparation for the treatment of a disease of the human or animal body selected from the group of chronic inflammatory intestinal diseases which is described in detail hereinabove.

### EXAMPLE

Detection of SLPI in Intestinal Samples from Healthy and Diseased Patients

Intestinal tissue samples of healthy and diseased patients were endoscopically recovered and immediately transferred to embedding medium for frozen sections (OCT, Miles Scientific) and then frozen in liquid nitrogen. Frozen sections were produced from the thus embedded samples and were immunologically stained.

For staining, the thus frozen sections were air-dried overnight and then fixed for 10 minutes with acetone-methanol-formaldehyde (AMF) at room temperature. The fixed frozen sections were then washed three times for 5 min each time with Tris-HCl buffer, pH value 7.4-7.6. The sections were then subjected to serum blocking for 30 minutes. Thereafter the sections were incubated with a first antibody (polyclonal rabbit antibody against human SLPI) in a dilution of 1:1,000 to 1:2,000 for 1 hour at 37° C. After washing three times with Tris-HCl buffer, pH value 7.4-7.6, for 5 minutes each time, the sections were incubated with a second antibody (biotinated goat anti-rabbit antibody); Vector ABC Kit) for 30 minutes at room temperature. After this the sections were again washed three times, as previously described. Then followed a 30-minute incubation with Vector ABC reagent. After this alkaline phosphatase was developed with substrate (Victor Red Alkaline Phosphatase Substrate Kit I). The color development was stopped with tap water after 20-30 minutes, and a 10-minute washing with tap water was performed. Counterstaining with 0.1% hematoxylin was then performed for 10 minutes. Excess stain was removed by a 10-minute washing with tap water. The sections were air-dried, covered and evaluated.

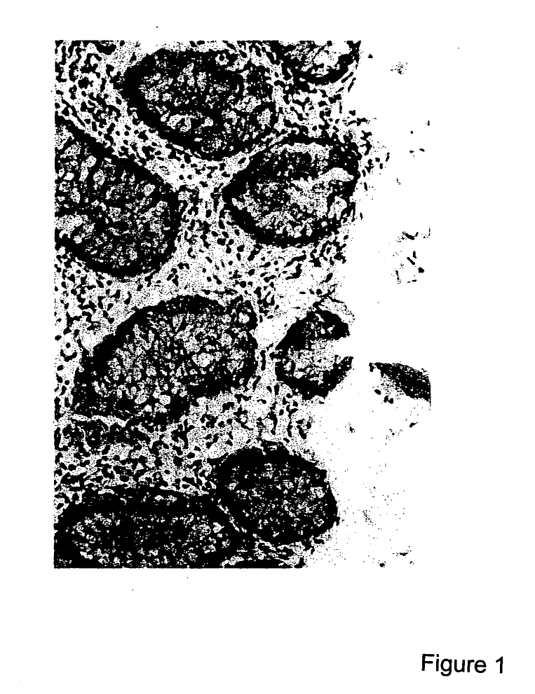

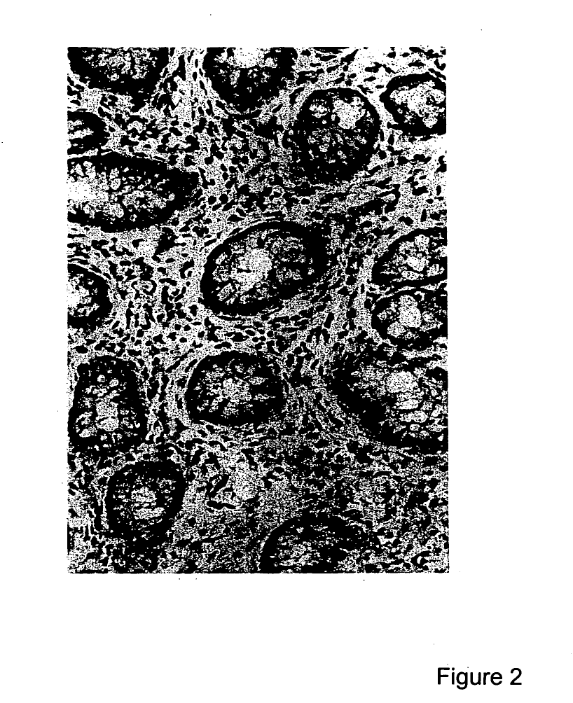

As for example is also to be seen in FIG. 1 (particularly the lower two cells), the cells of the intestinal mucosa of healthy subject are intensely colored (in the original photo: red). This intense red coloration shows that SLPI is present in very large amounts in the intestinal mucosa of healthy subjects. In contrast thereto, the cells of the intestinal mucosa of chronic inflammatory intestinal disease patients are scarcely colored (see FIG. 2). This slight coloration shows that the amount of SLPI in the intestinal mucosa of chronic inflammatory intestinal disease patients is strongly reduced. This observed strong reduction of the SLPI amount in the intestinal mucosa of chronic inflammatory intestinal disease patients is an indication of the suitability of SLPI for therapy of chronic inflammatory intestinal diseases.

1. Use of a effective material selected from the group consisting of secretory leucocyte protease inhibitor (SLPI), a fragment thereof, a complex thereof, a derivative thereof, an analog thereof, an expressible nucleic acid coding for the effective material SLPI or a fragment or derivative thereof, and a non-pathogenic microorganism containing the nucleic acid and capable of SLPI formation, for the treatment of a disease of a human or animal body, selected from the group of chronic inflammatory intestinal diseases consisting of enteritis necroticans, enteritis regionalis Crohn (Crohn's disease), colitis cystica, colitis granulomatosa, colitis gravis, colitis haemorrhagia, colitis ischaemica, colitis mucosa and colitis ulcerosa (ulcerative colitis).

2. Use according to claim 1, wherein the treatment takes place by the administration of the isolated and purified effective material in a pharmaceutical composition.

3. Use according to claim 2, wherein the effective material is administered in a dose which is sufficient to heal the chronic inflammatory intestinal disease state or to prevent it, to stop the progression of chronic inflammatory intestinal disease and/or to alleviate the chronic inflammatory intestinal disease symptoms.

4. Use according to claim 2 or 3, wherein the effective material is administered once through three times daily in a dose of 1-5,000 mg of effective material.

5. Use according to one of claims 2-4, wherein the effective material is administered orally.

6. Use according to claim 5, where in the effective material is administered in the form of a suspension, tablet, pill, capsule, lollipop, granulate or powder.

7. Use according to one of claims 2-4, wherein the effective material is administered rectally.

8. Use according to claim 7, wherein the effective material is administered in the form of a suppository, enema or foam.

9. Use according to one of claims 2-4, wherein the effective material is administered parenterally.

10. Use according to claim 9, wherein the effective material is administered in the form of an injection or infusion.

11. Use according to claim 1, wherein the non-pathogenic microorganism is capable of producing the effective material before, during or after administration to a human or animal and to release the produced effective material after administration to the organs of the digestive tract.

12. Use according to claim 11, wherein the non-pathogenic microorganism is a bacterial or fungal microorganism which belongs to the commensals of humans or animals.

13. Use according to claim 12, wherein the fungal microorganism belongs to the genus Saccharomyces.

14. Use according to claim 11, wherein the fungal microorganism is Saccharomyces boulardii.

15. Use according to claim 12, wherein the non-pathogenic microorganism belongs to the natural intestinal flora of humans or animals.

16. Use according to claim 15, wherein the non-pathogenic microorganism is an aerobic or anaerobic gram-negative bacterium of the intestinal flora.

17. Use according to claim 16, wherein the gram-negative bacterium belongs to the genus Escherichia, Pseudomonas, Bacteroides, or Proteus.

18. Use according to one of claims 17 [sic], wherein the gram-negative bacterium is Escherichia coli (Nissle 1917).

19. Use according to claim 15, wherein the non-pathogenic microorganism is an aerobic or anaerobic gram-positive bacterium of the intestinal flora.

20. Use according to claim 19, wherein the gram-positive bacterium belongs to the genus Bifidobacterium, Streptococcus, Staphylococcus, or Corynebacterium.

21. Use according to claim 20, wherein the gram-positive bacterium is Streptococcus gordonii.

22. Use according to claim 11, wherein the non-pathogenic microorganism is a microorganism which does not belong to the commensals of humans or animals.

23. Use according to claim 22, wherein the non-pathogenic microorganism is a bacterium which is used for the fermentative production of foodstuffs.

24. Use according to claim 22 or 23, wherein the bacterium concerned is a lactic acid bacterium, such as Lactococcus lactis, Lactobacillus delbrueckii subspec. bulgaricus, Lactobacillus casei, Lactobacillus caucasicus, Lactobacillus kefir, Streptococcus thermophilus, or Leconostoc.

25. Use according to one of claims 11-24, wherein a “leaky” mutant is concerned as the microorganism.

26. Use according to claim 11-25, wherein the nucleic acid coding for SLPI or a fragment or derivative thereof is inserted into a vector.

27. Use according to claim 26, wherein the vector is a plasmid, cosmid, bacteriophage or virus.

28. Use according to claim 26 or 27, wherein the nucleic acid inserted into a vector is under the functional control of at least one regulating element, which ensures the transcription of the nucleic acid in a translatable RNA and/or the translation of the RNA into a protein, before, during or after the administration.

29. Use according to claim 28, wherein the at least one regulating element is a promoter, a ribosome binding site, a signal sequence or a 3′-transcription terminator.

30. Use according to claim 29, wherein the promoter is an inducible promoter.

31. Use according to claim 30, wherein the promoter is a promoter which is inducible by nutrient deficiency.

32. Use according to claim 30 or 31, wherein the promoter is a trp-, lac- or tac-promoter of Escherichia coli.

33. Use according to claim 30 or 31, where in the promoter is the phoA promoter of Escherichia coli,

34. Use according to claim 30 or 31, wherein the promoter is the promoter of the PHO 5 gene of yeast.

35. Use according to claim 30 or 31, wherein the promoter is the promoter of the ADH 1 gene of yeast.

36. Use according to one of claims 29-35, wherein the ribosome binding site is a Shine-Dalgarno sequence.

37. Use according to one of claims 29-36, wherein the signal sequence is a bacterial or fungal signal sequence, which effects the secretion of the protein out of the cytoplasm of the microorganism into the periplasmic space or into the environment of the microorganism.

38. Use according to claim 37, wherein, as the bacterial signal sequence, the signal sequence of the β-lactamase gene of Escherichia coli or the signal sequence of the ompA gene of Escherichia coli is concerned.

39. Use according to claim 37, wherein, as the fungal signal sequence, the signal sequence of the α-factor of yeast or the signal sequence of the killer toxin of yeast is concerned.

40. Use according to one of claims 11-39, wherein the non-pathogenic microorganism capable of SLPI formation is contained in a pharmaceutical composition.

41. Use according to claim 40, wherein the pharmaceutical composition containing the microorganism is administered orally.

42. Use according to claim 41, wherein the pharmaceutical composition containing the microorganism is administered in the form of a suspension, tablet, pill, capsule, granulate or powder.

43. Use according to claim 40, wherein the pharmaceutical composition containing the microorganism is administered rectally.

44. Use according to claim 43, wherein the pharmaceutical composition containing the microorganism is administered in the form of a suppository, enema, or foam.

45. Pharmaceutical composition, comprising at least one cell of a non-pathogenic microorganism capable of forming SLPI and containing an expressible nucleic acid coding for SLPI or a fragment or derivative thereof.

46. Pharmaceutical composition according to claim 45, w herein the microorganism is an anaerobic or aerobic, gram-negative or gram-positive, bacterium of the intestinal flora.

47. Pharmaceutical composition according to claim 45, wherein the microorganism is a commensal yeast of humans or animals.

48. Pharmaceutical composition according to claim 45, wherein the microorganism is a bacterium which can be used for the fermentative production of foodstuffs.

49. Pharmaceutical composition according to claim 45, wherein the microorganism is a “leaky” mutant.

50. Pharmaceutical composition according to one of claims 45-49, wherein a nucleic acid coding for SLPI or a fragment or derivative thereof is inserted into an expression vector, and wherein the expression of the nucleic acid is under the control of at least one regulating element, so that the effective material is expressed before, during or after the administration of the pharmaceutical composition, and is released to the organs of the digestive tract after the administration of the pharmaceutical composition.

51. Method of production of a pharmaceutical composition, comprising:
(a) isolation or synthesis of a nucleic acid coding for the effective material SLPI;
(b) cloning of the nucleic acid coding for SLPI in a bacterial expression vector or a fungal expression vector;
(c) transformation of the recombinant expression vector obtained in (b) in a microbial host cell, where the host cell is a commensal of the human or animal intestinal flora and/or can be used for the fermentative production of foodstuffs;
(d) propagation of the transformed host cells;
(e) production of an immobilized, lyophilized, or liquid preparation of transformed host cells;
(f) mixing the immobilized, lyophilized, preparation or suspension of transformed host cells obtained in (e) with physiologically acceptable excipients, stabilizers, thickeners, parting agents, lubricants, emulsifiers or the like materials to obtain a pharmaceutical composition.
